(19) United States
(12) Patent Application Publication
(10) Pub. No.: US 2020/0005906 A1
(43) Pub. Date: Jan. 2, 2020

(54) CLINICAL TRIAL SEARCHING AND MATCHING

(71) Applicant: International Business Machines Corporation, Armonk, NY (US)

(72) Inventors: Fang Wang, Plano, TX (US); Jeff J. Li, Parkland, FL (US); Jia Xu, Somerville, MA (US); Vanessa Michelini, Boca Raton, FL (US); Kathleen A. Mancuso, Boston, MA (US)

(21) Appl. No.: 16/019,581

(22) Filed: Jun. 27, 2018

(51) Int. Cl. G16H 10/20; G16H 10/60; G06F 17/30

(52) U.S. Cl. CPC G16H 10/20; G16H 10/60; G06F 17/30327; G06F 17/30595; G06F 17/30867

(57) ABSTRACT

Embodiments describe an approach for improving eligibility criteria matching for clinical trials, the method comprising searching one or more proposed clinical trials, wherein the one or more proposed clinical trials comprises: a condition group, an intervention group and inclusion/exclusion criteria in the hierarchy structure. Determining if a patient's clinical information matches the one or more proposed clinical trial data. Responsive to determining a match between the patient clinical information matching and the one of the one or more proposed clinical trial data, wherein the matching comprises parent and child relationships for one or more patient clinical information, creating an entry in a clinical trial database based on the one or more proposed clinical trials and the patient clinical information, and outputting one or more clinical trials that match the patient clinical information in a structured format.

### BACKGROUND OF THE INVENTION

Clinical trials study whether medical procedures, drugs, or devices are safe and effective for treating patients. In oncology, doctors can use clinical trials as a method for providing cancer patients with new treatments to improve quality of life and extend patient survival. Currently, clinical trials are described in an unstandardized free-form text format in registries such as clinicaltrials.gov, which is the official clinical trial registry site for all US trials and some international trials. Each trial is broken down into several sections including title, condition, intervention, brief summary, current primary outcome, and study arms, but the text that goes within these sections is manually provided by the investigator in a free-form field. A clinical trial description can be very complex, involving many diseases, interventions, inclusive and exclusive eligibility criteria. Searching and finding clinical trials appropriate for a cancer patient is very challenging and inefficient because of the free-form structure, which can lead to many cancer patients missing out on potentially lifesaving treatment. In clinical trials, requirements that must be met for a person to be included in a trial are known as eligibility criteria. These requirements (e.g., eligibility criteria) help ensure that participants in a trial are like each other in terms of specific factors such as age, type and stage of cancer, general health, and previous treatment. When all participants meet the same eligibility criteria, it is more likely that results of the study are caused by the intervention being tested and not by other factors or by chance.

For example, to describe the same condition there are various ways of documenting it in clinicaltrials.gov, including abbreviations and the full name written out. Because the fields are all manually provided and there is no validation on free text, therefore typing errors are inevitable, increasing the difficulty to search. Additionally, embodiments of the present invention can match a disease (e.g., cancer) without locating/matching the exact name by using parent and child relationship between the definition, category, and/or treatment of one or more cancers and/clinical trials. Often, oncologists need to search by different keywords and synonyms to be able to capture most of the trials in scope and yet it is still not comprehensive. Additionally, the eligibility criteria of a clinical trial are too complicated to be simply satisfied by key word search. Once a trial is identified by keyword searches, they need to scrutinize all the eligibility criteria to see if the patient fulfills the requirements. Since the information in a trial covers lots of details and cannot be stored in a meaningful way, this manual process is then repeated again for another patient even though it has been reviewed before.

This manual approach is how it is performed nowadays. It is not efficient nor comprehensive. Most of the time, an oncologist's workload does not allow them to go through the trials in detail, thus patients are not enrolled in the most beneficial trial or in no trial at all, and simultaneously trials are not completed because they are not getting enough patient enrollment.

### SUMMARY

Embodiments of the present invention disclose a method, a computer program product, and a system for improving eligibility criteria matching for clinical trials. A method for improving eligibility criteria matching for clinical trials, the method comprising searching, by one or more processors, one or more proposed clinical trials, wherein the one or more proposed clinical trials comprises: a condition group and an intervention group. Determining, by the one or more processors, if a patient's clinical information matches the one or more proposed clinical trial data. Responsive to determining a match between the patient clinical information and the one of the one or more proposed clinical trial data, wherein the matching comprises parent and child relationships for one or more patient clinical information, creating, by one or more processors, an entry in a clinical trial database based on the one or more proposed clinical trials and the patient clinical information, and outputting, by the one or more processors, one or more clinical trials that match the patient clinical information in a structured format.

A computer system for improving eligibility criteria matching for clinical trials, the computer system comprising: one or more computer processors, one or more computer readable storage devices. Program instructions stored on the one or more computer readable storage devices for execution by at least one of the one or more computer processors, the stored program instructions comprising program instructions to search one or more proposed clinical trials, wherein the one or more proposed clinical trials comprises: a conditional group and an intervention group. Program instructions to determine if a patient's clinical information matches the one or more proposed clinical trial data. Responsive to determining a match between the patient clinical information and the one of the one or more proposed clinical trial data, wherein the matching comprises parent and child relationships for one or more patient clinical information program instructions to create an entry in a clinical trial database based on the one or more proposed clinical trials and the patient clinical information, and program instructions to output one or more clinical trials that match the patient clinical information in a structured format.

A computer program product for improving eligibility criteria matching for clinical trials, the computer program product comprising: one or more computer readable storage devices and program instructions stored on the one or more computer readable storage devices, the stored program instructions comprising program instructions to search one or more proposed clinical trials, wherein the one or more proposed clinical trials comprises: a conditional group and an intervention group. Program instructions to determine if a patient's clinical information matches the one or more proposed clinical trial data. Responsive to determining a match between the patient clinical information and the one of the one or more proposed clinical trial data, wherein the matching comprises parent and child relationships for one or more patient clinical information, program instructions to create an entry in a clinical trial database based on the one or more proposed clinical trials and the patient clinical information, and program instructions to output one or more clinical trials that match the patient clinical information in a structured format.

### BRIEF DESCRIPTION OF THE DRAWINGS

FIG. 1 is a functional block diagram illustrating a distributed data processing environment, in accordance with an embodiment of the present invention;

FIG. 2 illustrates an example of clinical trial mapping by clinical trial matching component 122, on a computing device within the distributed data processing environment of FIG. 1, in accordance with an embodiment of the present invention;

FIG. 3A depicts an organizational data structure of clinical trial matching in accordance with an embodiment of the present invention;

FIG. 3B depicts one example of clinical trial matching in accordance with an embodiment of the present invention;

FIG. 3C depicts one example of clinical trial matching in accordance with an embodiment of the present invention;

FIG. 4 illustrates operational steps of clinical trial matching component 122, on a computing device within the distributed data processing environment of FIG. 1, in accordance with an embodiment of the present invention;

FIG. 5 depicts one implementation of a database schema in accordance with an embodiment of the present invention;

FIG. 6 illustrates operational steps of clinical trial matching component 122, on a computing device within the distributed data processing environment of FIG. 1, in accordance with an embodiment of the present invention; and

FIG. 7 depicts a block diagram of components of the server computer executing the intelligent mapping program within the distributed data processing environment of FIG. 1, in accordance with an embodiment of the present invention.

### DETAILED DESCRIPTION

There is a need to improve clinical trial matching. Embodiments of the present invention enable a way to document clinical trials in a more structured format, suitable for searching and matching based on the patient's disease, patient's genetic makeups (biomarkers), and other relevant information in a more efficient and effective way. Embodiments of the present invention improve the storage of the documented trials enables an upfront review of the trials that could subsequently be applied to all relevant patient populations. This format is not limited by disease, intervention, or type of eligibility criteria and therefore could be used in the future as a standard structure for storing information for all clinical trials. Embodiments of the present invention can use “Clinical Trial Matching” (CTM) that uses Natural Language Processing (NLP) to extract information from both the clinical trials and the patient electronic records to match patients with the most relevant trials that fits a patient's unique/particular condition. Embodiments of the present invention are not here to discuss how the information is extracted or where the information is coming from.

The major differences between embodiments of the present invention and existing CTM's are mainly on how to describe clinical trials, categorize/organize, and how to perform the search; essentially improving the structure and organization of clinical trials in order to match patients with the best potentially lifesaving clinical trial efficiently. Embodiments of the present invention enables users to detect and store complicated information and relationships, including criteria for experimental arms, criteria related to biomarkers, criteria related to specific cancer types or interventions, and the prioritization of combined interventions. Embodiments of the present invention are structured so that it can include basket trials, umbrella trials, or any other trials known in the art. Existing/current CTM's do not store the arms of clinical trials, the interventions of clinical trials, and their relationships. Additionally, current CTM's has a very limited support of biomarkers.

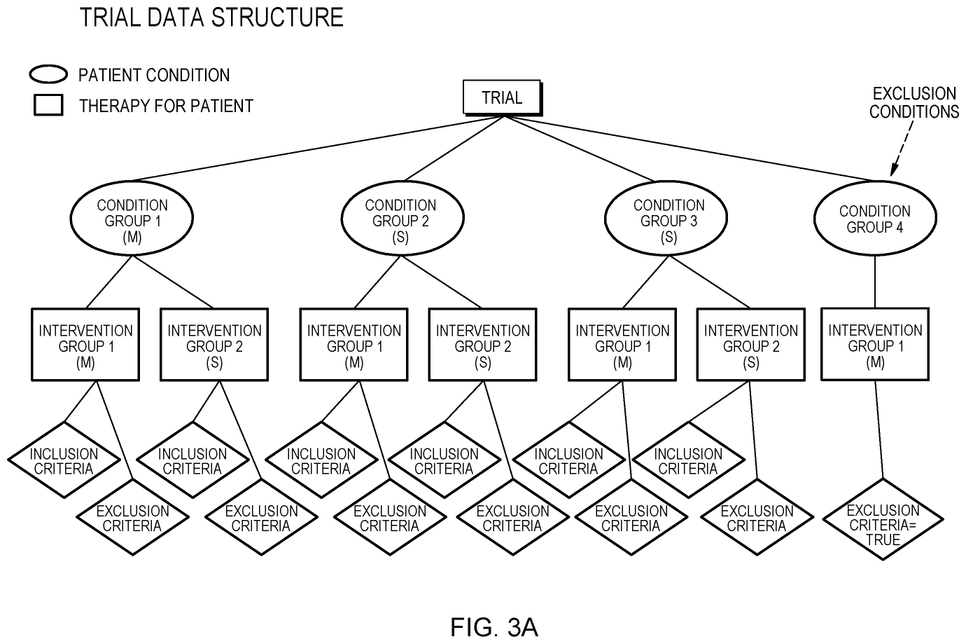

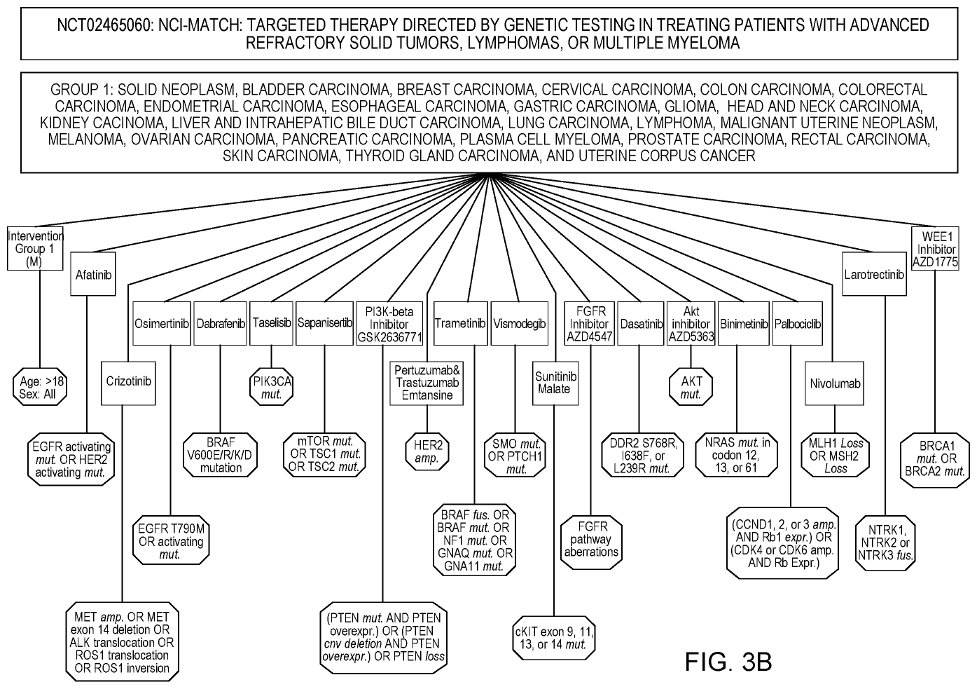

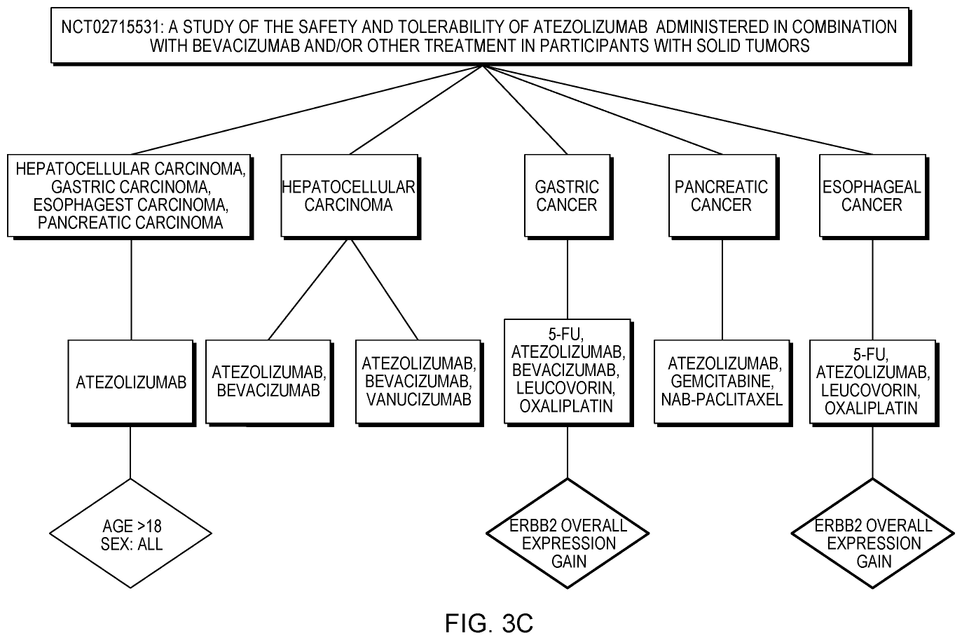

Embodiments of the present invention provide a well-defined hierarchical structure and unambiguous definition of clinical trials, as illustrated in FIG. 3A which solves the issue flat clinical trial data structures currently being used in the art. The flat clinical trial data structures cannot handle complicated scenarios with multiple arms. Embodiments of the present invention also solve the time-consuming task hand matching clinical trials that falls on the clinicians who don't have time to do. With the proposed tree structure, it is comprehensive enough to eliminate this repetitive manual work by fetching the trials in a precise and efficient way.

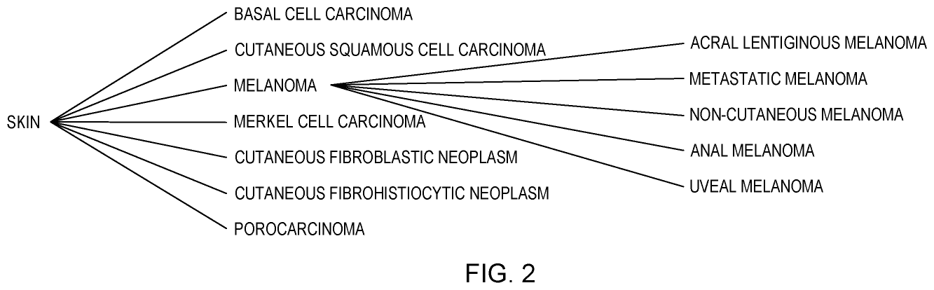

Additionally, disease condition codes and relationships are standardized in embodiments of the present invention so that embodiments of the present invention can search clinical trials based on the disease condition code and the relationships among condition codes, which solves an issue in the art. Currently, existing CTM's require an exact match of the term for the condition match. Embodiments of the present invention enable a one-time search that will provide an accurate, precise and comprehensive list of matching clinical trials, wherein an accurate, precise, and/or comprehensive list is based on a predetermined range, predetermined value, and/or predetermined degree of error in criteria matching between cancer types, condition groups (e.g., patient clinical information), and/or clinical trials, so that the practitioners (e.g., Medical Professionals) do not need to spend enormous amount of time on searching the clinical trial list on their own, as illustrated in FIG. 3B and FIG. 3C, which solves an problem in the art. For example, a predetermined range, value, and/or degree of error can be determined by a researcher to be twice removed from the main branch from the tree as illustrated in FIG. 2. Thus, embodiments of the present invention improve clinical trial matching, which in turn can improve clinical patient treatment by matching them to the most beneficial and potentially lifesaving clinical trial. Additionally, embodiments of the present invention improve eligibility criteria matching for trials and provide an improved approach for describing clinical trials in a tree structured and/or umbrella structured format, as shown in FIG. 2, and FIG. 3A-3C, to replace the existing and problematic free-text format. The structure format enables users to search clinical trials efficiently using patient's diseases, age, country codes, biomarkers, and other information and provide more accurate results for clinical trial searches. The embodiments shown in FIG. 2, and FIG. 3A-3C are embodiments of complicated cases that the current flat structure's used in the art cannot handle. Complicated cases are becoming more and more common, as technology and medicine advances and the advancing knowledge of how important the role of a patient's genetic makeup is to drug response.

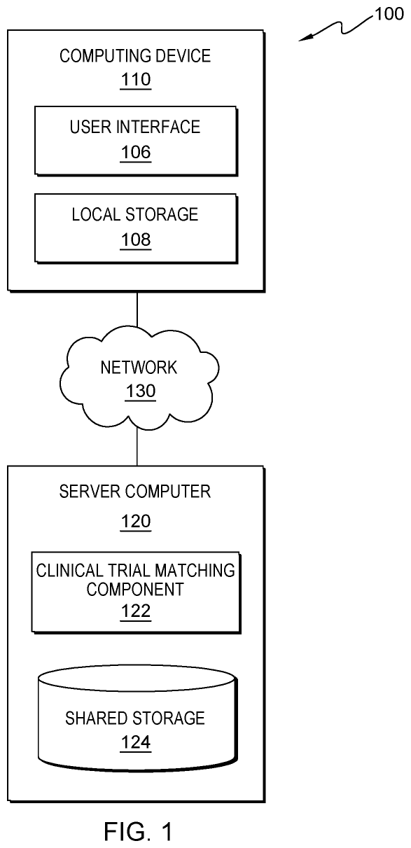

Embodiments of the present invention address a need for clinical trials matching capable of returning eligible trials where the patient cancer type or disease type is not an exact match for any of the conditions listed in the trial. When matching trials by hand, search term, or some alternative trial matching software, only trials where the search term matches the condition that is listed are returned. For example, when searching for melanoma using this term in clinicaltirals.gov, a clinical trial website (government and/or private), only trials that specifically list melanoma in the condition are returned. In this particular example, by incorporating cancer codes and mapping methods established show the relationships between cancer types, embodiments of the present invention can automatically identify and/or retrieve trials with terms related to the search including both parent and child relationships for each condition, which improves the art and solves the problems of the current CTM models. Embodiments of the present invention improve the art of clinical trial matching by improving clinical trial structure, categorization, organization, and storage, which makes search more efficient and effective. Embodiments of the present invention improve the art of clinical trial matching (i.e., how to perform the clinical trial search/matching trials with patient clinical information) by using NLP to extract data/information from both clinical trials and patient electronic records to match patients with the most relevant trials that fits a patient's unique/particular clinical information, and improving the art of clinical trial matching by detecting and storing complicated information and relationships, including criteria for experimental arms, criteria related to biomarkers, criteria related to specific disease, illness, and cancer types or interventions, and the prioritization of combined interventions. FIG. 1 is a functional block diagram illustrating a distributed data processing environment, generally designated 100, in accordance with one embodiment of the present invention. The term “distributed” as used in this specification describes a computer system that includes multiple, physically distinct devices that operate together as a single computer system. FIG. 1 provides only an illustration of one implementation and does not imply any limitations with regard to the environments in which different embodiments can be implemented. Many modifications to the depicted environment can be made by those skilled in the art without departing from the scope of the invention as recited by the claims.

Distributed data processing environment 100 includes computing device 110, server computer 120, interconnected over network 130. Network 130 can be, for example, a telecommunications network, a local area network (LAN), a wide area network (WAN), such as the Internet, a wireless technology for exchanging data over short distances (using short-wavelength ultra high frequency (UHF) radio waves in the industrial, scientific and medical (ISM) band from 2.4 to 2.485 GHz from fixed and mobile devices, and building personal area networks (PANs) or a combination of the three, and can include wired, wireless, or fiber optic connections. Network 130 can include one or more wired and/or wireless networks that are capable of receiving and transmitting data, voice, and/or video signals, including multimedia signals that include voice, data, text and/or video information. In general, network 130 can be any combination of connections and protocols that will support communications between computing device 110 and server computer 120, and other computing devices (not shown in FIG. 1) within distributed data processing environment 100.

In various embodiments, computing device 110 can be, but is not limited to, a standalone device, a server, a laptop computer, a tablet computer, a netbook computer, a personal computer (PC), a smart phone, a desktop computer, a smart television, a smart watch, a radio, stereo system, a cloud based service (e.g., a cognitive cloud based service), and/or any programmable electronic computing device capable of communicating with various components and devices within distributed data processing environment 100, via network 130 or any combination therein. In general, computing device 110 are representative of any programmable mobile device or a combination of programmable mobile devices capable of executing machine-readable program instructions and communicating with users of other mobile devices via network 130 and/or capable of executing machine-readable program instructions and communicating with server computer 120. In other embodiments, computing device 110 can represent any programmable electronic computing device or combination of programmable electronic computing devices capable of executing machine readable program instructions, manipulating executable machine readable instructions, and communicating with server computer 120 and other computing devices (not shown) within distributed data processing environment 100 via a network, such as network 130. Computing device 110 includes an instance of user interface 106. Computing device 110 and user interface 106 allow a user to interact with clinical trial matching component (CTMC) 122 in various ways, such as sending program instructions, receiving messages, sending data, inputting data, editing data, correcting data and/or receiving data. In various embodiments, not depicted in FIG. 1, computing device 110 can have one or more user interfaces. In other embodiments, not depicted in FIG. 1 environment 100 can comprise one or more computing devices (e.g., at least two).

User interface (UI) 106 provides an interface to CTMC 122 on server computer 120 for a user of computing device 110. In one embodiment, UI 106 can be a graphical user interface (GUI) or a web user interface (WUI) and can display text, documents, web browser windows, user options, application interfaces, and instructions for operation, and include the information (such as graphic, text, and sound) that a program presents to a user and the control sequences the user employs to control the program. In another embodiment, UI 106 can also be mobile application software that provides an interface between a user of computing device 110 and server computer 120. Mobile application software, or an “app,” is a computer program designed to run on smart phones, tablet computers and other mobile devices. In an embodiment, UI 106 enables the user of computing device 110 to send data, input data, edit data (annotations), correct data and/or receive data.

Server computer 120 can be a standalone computing device, a management server, a web server, a mobile computing device, or any other electronic device or computing system capable of receiving, sending, and processing data. In other embodiments, server computer 120 can represent a server computing system utilizing multiple computers as a server system, such as in a cloud computing environment. In another embodiment, server computer 120 can be a laptop computer, a tablet computer, a netbook computer, a personal computer (PC), a desktop computer, a personal digital assistant (PDA), a smart phone, or any other programmable electronic device capable of communicating with computing device 110 and other computing devices (not shown) within distributed data processing environment 100 via network 130. In another embodiment, server computer 120 represents a computing system utilizing clustered computers and components (e.g., database server computers, application server computers, etc.) that act as a single pool of seamless resources when accessed within distributed data processing environment 100. Server computer 120 can include internal and external hardware components, as depicted, and described in further detail with respect to FIG. 3.

Shared storage 124 and local storage 108 can be a data repository and/or a database that can be written to and/or read by one or a combination of CTMC 122, server computer 120 and/or computing device 110. In the depicted embodiment, shared storage 124 resides on server computer 120. In another embodiment, shared storage 124 can reside elsewhere within distributed data processing environment 100 provided coverage assessment program 110 has access to shared storage 124. A database is an organized collection of data. Shared storage 124 and/or local storage 108 can be implemented with any type of storage device capable of storing data and configuration files that can be accessed and utilized by server computer 120, such as a database server, a hard disk drive, or a flash memory. In other embodiments, shared storage 124 and/or local storage 108 can be hard drives, memory cards, computer output to laser disc (cold storage), and/or any form of data storage known in the art.

In some embodiments, shared storage 124 and/or local storage 108 can be cloud storage systems and/or databases linked to a cloud network. In various embodiments, CTMC 122 can search, identify, match, and/or retrieve clinical trials from a clinical trial database (e.g., shared storage 124 and/or local storage 108) that match a patient's clinical information. For example, CTMC 122 will search and/or store patient clinical information, patient electronic records, clinical trials, and/or clinical trial matches to shared storage 124, in which CTMC 122 can access at a later time to either reuse and/or expedite the matching patients with similar patient clinical information. In this particular example, CTMC 122 can create a database based on the stored clinical, patient clinical information, patient electronic records, clinical trials, and/or clinical trial matches. Patient clinical information can be, but are not limited to, overall information about a patient such as: age, gender, cancer condition, genomic info (with respect to mutations), medical history, family medical history, type of illness, allergies, biomarker data, type of disease, length of illness and/or disease, cause of illness or disease, physical fitness, socioeconomic status, nationality, genetic make-up, genetic response to medication, genetic predisposition, and/or any other patient, medical, illness, and/or disease data known in the art. Patient clinical information and other personal patient data know in the art can be stored within patient electronic records. Eligibility criteria can be the corresponding information specified in the clinical trials.

In various embodiments, CTMC 122 can use “Clinical Trial Matching” (CTM) that uses Natural Language Processing (NLP) to extract information from both the clinical trials and the patient electronic records to match patients with the most relevant trials that fits a patients particular needs. In various embodiments, CTMC 122 can enables one or more users to detect and store complicated information and relationships, including criteria for trial arms, criteria related to biomarkers, criteria related to specific cancer types or interventions, and the prioritization of combined interventions, as depicted in FIG. 3A. In some embodiments, CTMC 122 can be structured so that it can include basket trials, umbrella trials, or any other trials known in the art. In various embodiments, CTMC 122 can store the arms of clinical trials, the interventions of clinical trials, and their relationships. In various embodiments, CTMC 122 can provide a well-defined structure and unambiguous definition of clinical trials, depicted in FIG. 3A-3C. Additionally, CTMC 122 can standardize disease condition codes and relationships so that CTMC 122 can base the clinical trials search on the disease condition code and the relationships among condition code. In various embodiments, CTMC 122 can incorporate cancer codes and mapping methods to show the relationships between cancer types, wherein CTMC 122 can automatically identify and/or retrieve trials with terms related to the search including both parent and child relationships for each condition. In some embodiments, CTMC 122 can automatically truncate and/or alter boollean operators and/or search terms (e.g., patient clinical information and/or clinical trial data) in order to optimize search results and improve clinical trial mapping.

In various embodiments, CTMC 122 can utilize disease condition codes and relationships, which are standardized, so that CTMC 122 can search clinical trials based on the disease condition code and the relationships among condition code efficiently and effectively, shown in FIG. 2. Additionally, in this particular embodiment, CTMC 122 can retrieve clinical trials from a database that fit a patients clinical information without finding an exact match, using the standardized disease condition codes and relationships to provide an accurate, precise and comprehensive list of matching clinical trials so that the users/practitioners (e.g., Medical Professionals) no longer need to waste amount of time searching the clinical trial list on their own. In other embodiments, CTMC 122 can provide a prioritized list of clinical trials ranking the clinical trials from closest match to the patient clinical information to the least closest match. In various embodiments, CTMC 122 can produce a structure format, as depicted in FIG. 3A, that enables users/practitioners to search clinical trials efficiently using patient's patient clinical information (e.g., diseases, age, country codes, biomarkers, and other medical and/or personal information known in the art) and provide accurate results for clinical trial searches.

For example, returning to a melanoma example, CTMC 122 can also return/retrieve clinical trials that have skin cancer listed as a condition, a parent relationship to melanoma, and/or any of the child relationships to melanoma, including Acral Lentiginous Melanoma, Metastatic Melanoma, etc (see FIG. 2). Additionally, in this particular example, CTMC 122 can also match melanomas trials that accept all solid tumors; a crucial result with the increased prevalence of umbrella studies is resulting from the development of new targeted therapies. In various embodiments, CTMC 122 can standardize cancer types and their relationships in a structure format, in which the relationships among the cancer types can be used during the clinical trial matching.

In the publicly available forms of the trials (i.e. clinicaltrials.gov), the information is not clearly divided, structured or searchable to enable accurate searches for trials based on patient criteria. The difficulties of searching these trials include that the current organization allows for eligible condition subtypes to be specified outside condition section, no standardized method for listing biomarker requirements, specifying the interventions to only be used for patients with specific conditions or biomarkers specified in the “Arms” section of the trial, and more. The lack of standardization and structure result list in patients not having a full, accurate list of trials they are eligible for as well as trials not reaching their necessary enrollment. CTMC 122 provides a standardized and structure result list. For example, as shown in FIG. 3A, a clinical trial is described using four types of data items: (i) Condition group. A condition group includes one to many cancer conditions such as gastric cancer, Heart Disease, Alzheimer's Disease, etc. Conditions are mapped using cancer codes, in order to match trials that have a correct parent or child relationship to the condition listed in the trial; (ii) intervention group, wherein an intervention group comprises one or more pharmaceutical drugs, devices or procedures, such as Atezolizumab, stent, counseling, etc.; (iii) Inclusive criteria for example, characteristics the patient must have, such as having EGFR T790M mutation, must be a female, and/or must be 18 years old; and (iv) exclusive criteria, for example, characteristics the patient must not have, such as the patient with a gain in their overall expression of ERBB2 will be excluded.

In various embodiments, a trial can be suitable for one or more condition groups, wherein one or more condition groups can be associated with one or many therapy groups, and where one or more therapy groups have one or more inclusive and/or exclusive criteria. As shown in FIG. 3B and FIG. 3C, CTMC 122 organizes and/or structures two clinical trials in the specified structure format using condition groups, interventions groups, and inclusive and exclusive criteria. For example, the clinical trial NCT02465060, as shown in FIG. 3B, clearly demonstrates some of the complexities that exist within trials, showing that in this Basket Trial many cancer types are included but in order to be treated with a specific intervention a specific biomarker is required. For example, in this particular trial a general inclusion criteria is that all patients must be older than 18 and an intervention specific criteria is that to be treated with Afatinib a patient must have an activating mutation in either EGFR or HER2. In this particular example, CTMC 122 can only locate and/or was instructed to only locate and/or identify inclusion criteria based on the intervention group, condition groups, and/or patient clinical information. In other embodiments, CTMC 122 can locate and identify inclusion criteria and/or exclusion criteria based on the intervention group, condition groups, and/or patient clinical information, and in some embodiments, CTMC 122 can locate and/or identify intervention groups that do not have inclusion criteria and/or exclusion criteria, based condition groups, and/or patient clinical information. For example, in FIG. 3C, CTMC 122 identifies the inclusion criteria for Atezolizumab is older than 18 years old, the exclusion criteria for 5-FU, Atezolizumab, Bevacizumab, Leucovorin, and/or Oxalipaltin is ERBB2 overall expression gain, and CTMC 122 identified/located 3 intervention groups that don't have inclusion criteria or exclusion criteria

In this particular example, CTMC 122 solves the issues presented above by defining clinical trials based on three main concepts: condition groups, intervention groups, and inclusion and/or exclusion criteria (e.g., clinical trial data). In this particular example, the tree main concepts are then incorporated into a tree structure to support efficient searching, which improves the key words based approach currently used in the art because embodiments of the current invention can be more flexible and it enables CTMC 122 to describe the complicated relationship between conditions, interventions and inclusive/exclusive criteria accurately. In various embodiments, CTMC 122 can be used to support filtering based on the information available in the patient info (e.g., patient clinical information), based on mutations available from molecular profile analysis, and/or based on a targeted drug, based on the logical relationships created on top of different types/kind of criteria, such as, but not limited to, prior treatment, brain metastasis, patient performance, measurable disease, tumor stage, lines of therapies etc. It should be noted that clinical trial criteria and proposed clinical trial criteria can be interchangeable. It should be noted that clinical trial and proposed clinical trial can be interchangeable. Proposed clinical trial can be a current, suggested, and/or historic clinical trial.

In various embodiments, CTMC 122 can provide the design for offering a cloud-based clinical trial searching service. The service input, via UI 106 can include, but is not limited to, the patient's disease, age, country codes, pharmaceutical compounds, and/or genetic mutations. In some embodiments, service input can be patient clinical information. In some embodiments, the service output is a list of clinical trials matching to the service input. In some embodiments, service consumers (e.g., Practitioners) can get an accurate list of trials based on the given information without needing to read and understand the nuances of other unsuitable clinical trials.

In various embodiments, CTMC 122 can utilize and/or identify use cases. In some embodiments, CTMC 122 can identify two or more use cases. In a particular embodiment, CTMC 122 identifies two use cases, in which one use case comprises: a search for clinical trials based on the patient clinical information such as disease, age, location, gene mutations, and other information/data; and the second use case comprises: a search for clinical trials available for particular drug and patient information. A drug can be very effective for a gene mutation but the FDA has not approved the drug for a certain disease, CTMC 122 can enable one or more practitioners to determine if there are clinical trials available for the drug suitable for the patient clinical information.

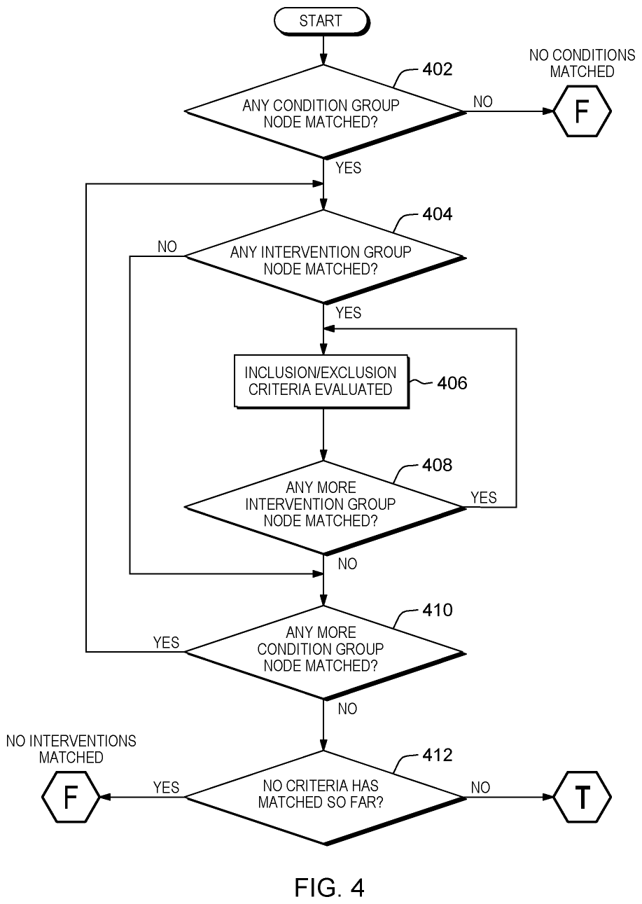

In various embodiments CTMC 122 can match clinical trials to patient clinical information. FIG. 4 illustrates one embodiment of the clinical trial searching process. In step 402, CTMC 122 determines if the Patient clinical information match with multiple condition group nodes. In this particular embodiment, if CTMC 122 determines there are no group nodes that match one or more of the patient clinical information then CTMC 122 can end the clinical trial searching process. However, in this particular embodiment, if CTMC 122 determines one or more group nodes match a patient clinical information (Yes branch), then CTMC 122 can advance to step 410 to determine if there are any more condition groups that match one or more of the patient clinical information and/or advance to step 404. In step 404, CTMC 122 determines if one or more intervention group nodes match one or more of a patient clinical information. In this particular embodiment, if CTMC 122 determines there are no intervention group nodes that match the patients one or more conditions (No branch) then CTMC 122 can advance to step 410.

In this particular embodiment, if CTMC 122 determines there are intervention group nodes that match a patients one or more conditions, then CTMC 122 can advance to step 406 and/or step 408. In this particular embodiment, under a condition group node, the therapy specified in a service input may match with multiple intervention groups. If the therapy is not specified in the input such as the first use case, a user via UI 106 and/or CTMC 122 uses “any” as the value of the specified therapy, “any” will match with all the invention groups under a condition group. In step 406, CTMC 122 records if the inclusion and/or exclusion criteria is evaluated and/or satisfied. For example, inclusion criteria can require a minimum age of 18, female, have EGFR L858R mutation and exclusion criteria can have certain drug as previous treatment. One or more satisfied criteria means inclusion criteria was matched with patient clinical information (after considering whatever logic operation the trial has asked for) and none of exclusion criteria was matched. Then the corresponding branch of condition group/intervention group combination is considered match. An inclusion or exclusion criteria may be false or true, depending on the patient bio-markers, age, country code, or other information. In another example, a clinical trial asking for patients carrying EGFR L858R mutation to be eligible for the trial. The patient's molecular profile detected from the sequencing needs to contain this mutation. If not, then the patient is not eligible for the trial.

In step 408, CTMC 122 determines if there are any more intervention group nodes that match queried therapies. In this particular embodiment, if CTMC 122 determine there are more intervention group nodes that match (Yes branch) then CTMC 122 can repeat steps 406-408. However, if CTMC 122 determines there are no more matches between the intervention group nodes and the queried therapies (No branch) then CTMC 122 can advance to step 410. In step 410, CTMC 122 determines if there are any more condition group nodes that match a patient clinical information. In this particular embodiment, if CTMC 122 determines there are more condition group nodes that match a patient clinical information (Yes branch) then CTMC 122 can repeat steps 404-410. However, in this particular embodiment, if CTMC 122 determines there are no more condition group matches (No branch) then CTMC 122 can advance to step 412. In step 412, CTMC 122 determines if no criteria matches a patient clinical information.

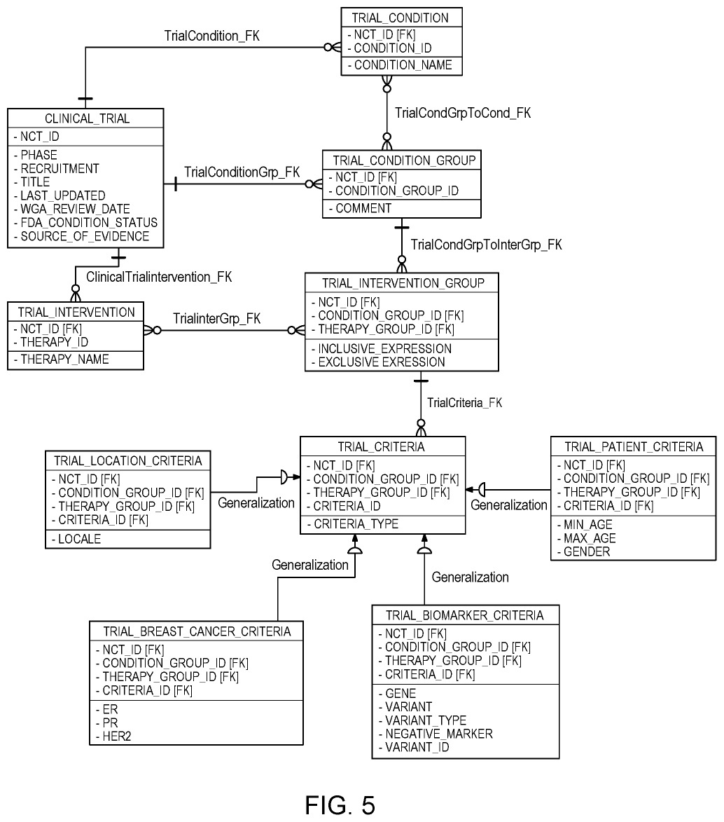

In this particular embodiment, if CTMC 122 determines that there is no criteria that matches a patient clinical information (Yes branch) then CTMC 122 can determine there are no matches/the search produces no results and the trial search process ends. However, in this particular embodiment, if CTMC 122 determines there is criteria that matches a patient clinical information (No branch) then the search is considered to be true and CTMC 122 can retrieve and/or produce the trials that match the patient clinical information. A trial can be considered a match if the inclusion criteria leaf nodes on one matched path (condition group/intervention group) are true and none of the exclusion criteria leaf nodes on that path are true. In the complicated cases, we can use a logical expression to describe the relationships among inclusive criteria/exclusive criteria and evaluate the logical expression to determine if we have a match on a path. In various embodiments, CTMC 122 can support criteria of many kinds, such as criteria based on the patient age, gender, and location, criteria based on condition types, and criteria based on the patient's gene mutation. For example, a relationships among criteria can be described in logical expressions such as criteria1 && criteria2 && (!criteria3∥!criteria4). In various embodiments, CTMC 122 can implement a database implementation schema, as shown in FIG. 5, that demonstrates how clinical trials can be stored in a relational database schema to support efficient search.

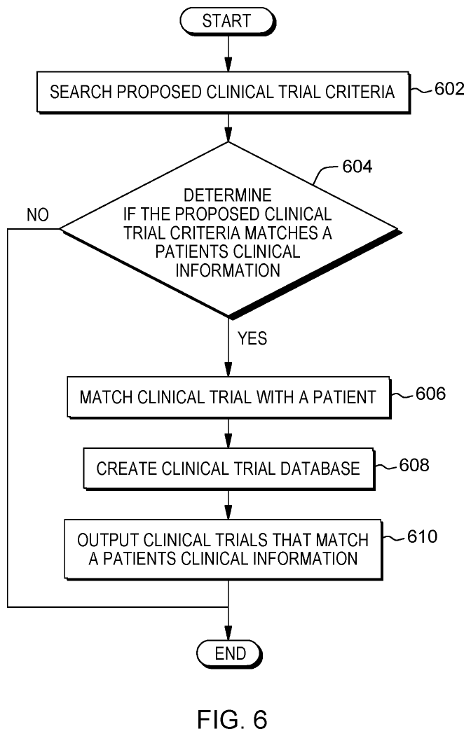

FIG. 6 is a flowchart depicting operational steps of CTMC 122, on server computer 120 within distributed data processing environment 100 of FIG. 1, in accordance with an embodiment of the present invention. It should be appreciated that FIG. 6 provides only an illustration of one implementation and does not imply any limitations with regard to the environments in which different embodiments can be implemented. Many modifications to the depicted environment can be made.

In step 602, CTMC 122 searches the proposed clinical trial criteria of clinical trials in a database. In various embodiments, CTMC 122 can search one or more of the proposed clinical trial criteria or clinical trials in a database (e.g., clinical trial database), wherein the proposed clinical trial criteria comprises: condition groups, intervention groups, and inclusion and/or exclusion criteria. In various embodiments, CTMC 122 can search the proposed clinical trial criteria of current and/or historic clinical trials that match a patient clinical information, wherein the matching of patient clinical information can be a subset match of one or more of the patient clinical information.

In step 604, CTMC 122 determines if the proposed clinical trial criteria matches one or more patient clinical information. In various embodiments, CTMC 122 can determine if one or more proposed clinical trial criteria in a database match one or more of a patient's one or more conditions, as shown in FIG. 4. In various embodiments, CTMC 122 can determine if there are one or more subgroup/related clinical trial criteria that matches one or more patient clinical information. In this particular embodiment, if CTMC 122 determines there are not matches either exact and/or related (No branch), then CTMC 122 can end the clinical trial search. However, in this particular embodiment, if CTMC 122 determines there are matches either exact and/or related (Yes branch), then CTMC 122 can proceed to step 606.

In step 606, CTMC 122 matches a clinical trial with a patient. In various embodiments, CTMC 122 can match and/or retrieve one or more clinical trials with one or more patients based on the matching and/or relationship between one or more patient clinical information and one or more proposed clinical trial data, via NLP. In various embodiments, step 604 and/or step 606 can comprise identifying clinical trial criteria from historic clinical trial data stored on a database. In step 608, CTMC 122 can create a clinical trial database based on the matched clinical trials and patient clinical information. In step 610, CTMC 122 can output clinical trial that matches a patient clinical information. In various embodiments, CTMC 122 can output one or more clinical trials based on one or more matched clinical trials to one or more patient clinical information. In one particular embodiment, CTMC 122 can output the one or more matched clinical trials to a user (e.g., Practitioner and/or Physician), via UI 106, and/or printed document. In other embodiments, CTMC 122 can automatically enroll a patient into a study if the clinical trial match is within a predetermined threshold.

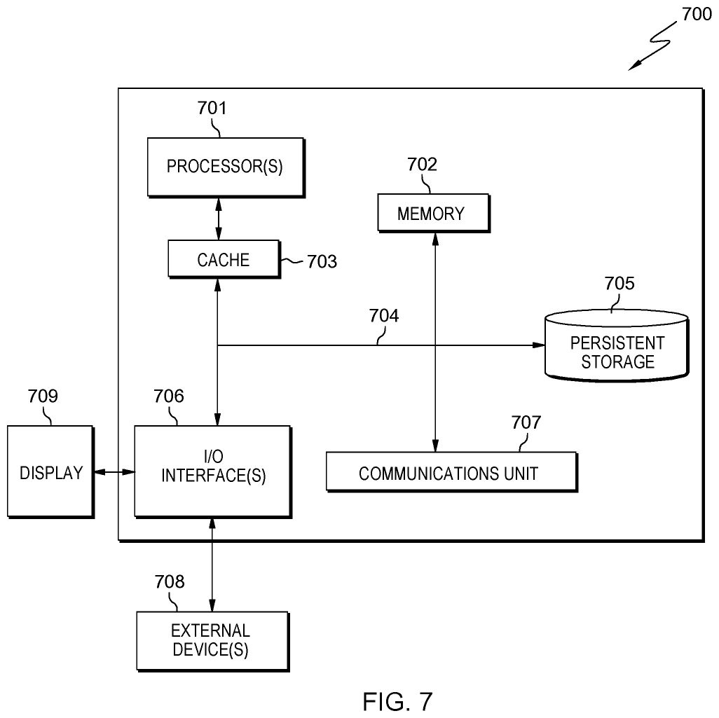

FIG. 7 depicts computer system 700, where server computer 120 represents an example of computer system 700 that includes CTMC 122. The computer system includes processors 701, cache 703, memory 702, persistent storage 705, communications unit 707, input/output (I/O) interface(s) 706 and communications fabric 704. Communications fabric 704 provides communications between cache 703, memory 702, persistent storage 705, communications unit 707, and input/output (I/O) interface(s) 706. Communications fabric 704 can be implemented with any architecture designed for passing data and/or control information between processors (such as microprocessors, communications, and network processors, etc.), system memory, peripheral devices, and any other hardware components within a system. For example, communications fabric 704 can be implemented with one or more buses or a crossbar switch.

Memory 702 and persistent storage 705 are computer readable storage media. In this embodiment, memory 702 includes random access memory (RAM). In general, memory 702 can include any suitable volatile or non-volatile computer readable storage media. Cache 703 is a fast memory that enhances the performance of processors 701 by holding recently accessed data, and data near recently accessed data, from memory 702.

Program instructions and data used to practice embodiments of the present invention can be stored in persistent storage 705 and in memory 702 for execution by one or more of the respective processors 701 via cache 703. In an embodiment, persistent storage 705 includes a magnetic hard disk drive. Alternatively, or in addition to a magnetic hard disk drive, persistent storage 705 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 705 can also be removable. For example, a removable hard drive can be used for persistent storage 705. Other examples include optical and magnetic disks, thumb drives, and smart cards that are inserted into a drive for transfer onto another computer readable storage medium that is also part of persistent storage 705.

Communications unit 707, in these examples, provides for communications with other data processing systems or devices. In these examples, communications unit 707 includes one or more network interface cards. Communications unit 707 can provide communications through the use of either or both physical and wireless communications links. Program instructions and data used to practice embodiments of the present invention can be downloaded to persistent storage 705 through communications unit 707.

I/O interface(s) 706 enables for input and output of data with other devices that can be connected to each computer system. For example, I/O interface 706 can provide a connection to external devices 708 such as a keyboard, keypad, a touch screen, and/or some other suitable input device. External devices 708 can also include portable computer readable storage media such as, for example, thumb drives, portable optical or magnetic disks, and memory cards. Software and data used to practice embodiments of the present invention can be stored on such portable computer readable storage media and can be loaded onto persistent storage 705 via I/O interface(s) 706. I/O interface(s) 706 also connect to display 709.

Display 709 provides a mechanism to display data to a user and can be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention can be a system, a method, and/or a computer program product. The computer program product can include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general-purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, a segment, or a portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A method for improving criteria eligibility matching for clinical trials, the method comprising:
searching, by one or more processors, one or more proposed clinical trials, wherein the one or more proposed clinical trials comprises: a condition group and an intervention group;
determining, by the one or more processors, if a patient clinical information matches the one or more proposed clinical trial data;
responsive to determining a match between the patient clinical information and the one of the one or more proposed clinical trial data, wherein the matching comprises parent and child relationships for one or more patient clinical information, creating, by one or more processors, an entry in a clinical trial database based on the one or more proposed clinical trials and the patient clinical information; and
outputting, by the one or more processors, one or more clinical trials that match the patient clinical information in a structured format.

2. The method of claim 1, wherein matching further comprises:
identifying, by the one or more processors, clinical trial criteria from historic clinical trial data that matches the patient clinical information.

3. The method of claim 1, wherein a clinical trial is considered a match if an inclusion criteria leaf nodes on one matched path are true and none of an exclusion criteria leaf nodes on the path are true.

4. The method of claim 1, wherein searching the one or more proposed clinical trials is based on standardized disease codes and relationship among the standardized disease codes.

5. The method of claim 1, wherein determining further comprises:
retrieving, by the one or more processors, clinical trials from a clinical trial database that matches a patient clinical information without finding an exact match, using a standardized disease condition codes and relationships to provide a of matching clinical trials.

6. The method of claim 1, wherein determining further comprises using natural language processing to extract data from both the one or more proposed clinical trials and the patient clinical information to match the patient with the clinical trial traits that fit the patient clinical information needs.

7. The method of claim 6, wherein patient clinical information comprise: type of illness, allergies, biomarker data, type of disease, length of illness, length of disease, cause of illness, cause of disease, medical history, family medical history, gender, physical fitness, age, socioeconomic status, nationality, genetic make-up, genetic response to medication, and genetic predispositions.

8. A computer system for improving eligibility criteria matching for clinical trials, the computer system comprising:
one or more computer processors;
one or more computer readable storage devices;
program instructions stored on the one or more computer readable storage devices for execution by at least one of the one or more computer processors, the stored program instructions comprising:
program instructions to search one or more proposed clinical trials, wherein the one or more proposed clinical trials comprises: a condition group and an intervention group;
program instructions to determine if a patient clinical information match the one or more proposed clinical trial data;
responsive to determining a match between the patient clinical information matching and the one of the one or more proposed clinical trial data, wherein the matching comprises parent and child relationships for one or more patient clinical information, program instructions to create an entry in a clinical trial database based on the one or more proposed clinical trials and the patient clinical information; and
program instructions to output one or more clinical trials that match the patient clinical information in a structured format.

9. The computer system of claim 8, wherein matching further comprises:
program instructions to identify clinical trial criteria from historic clinical trial data that matches the patient clinical information.

10. The computer system of claim 8, wherein a clinical trial is considered a match if an inclusion criteria leaf nodes on one matched path are true and none of an exclusion criteria leaf nodes on the path are true.

11. The computer system of claim 8, wherein searching the one or more proposed clinical trials is based on standardized disease codes and relationship among the standardized disease codes.

12. The computer system of claim 8, wherein determining further comprises:
program instructions to retrieve clinical trials from a clinical trial database that matches a patient clinical information without finding an exact match, using a standardized disease condition codes and relationships to provide a of matching clinical trials.

13. The computer system of claim 8, wherein determining further comprises using natural language processing to extract data from both the one or more proposed clinical trials and the patient clinical information to match the patient with the clinical trial traits that fit the patient clinical information needs.

14. The computer system of claim 13, wherein patient clinical information comprise: type of illness, allergies, biomarker data, type of disease, length of illness, length of disease, cause of illness, cause of disease, medical history, family medical history, gender, physical fitness, age, socioeconomic status, nationality, genetic make-up, genetic response to medication, and genetic predispositions.

15. A computer program product for improving eligibility criteria matching for clinical trials, the computer program product comprising:
one or more computer readable storage devices and program instructions stored on the one or more computer readable storage devices, the stored program instructions comprising:
program instructions to search one or more proposed clinical trials, wherein the one or more proposed clinical trials comprises: a condition group and an intervention group;
program instructions to determine if a patient clinical information match the one or more proposed clinical trial data;
responsive to determining a match between the patient clinical information matching and the one of the one or more proposed clinical trial data, wherein the matching comprises parent and child relationships for one or more patient clinical information, program instructions to create an entry in a clinical trial database based on the one or more proposed clinical trials and the patient clinical information; and
program instructions to output one or more clinical trials that match the patient clinical information in a structured format.

16. The computer program product of claim 15, wherein matching further comprises:
program instructions to identify clinical trial criteria from historic clinical trial data that matches the patient clinical information.

17. The computer program product of claim 15, wherein a clinical trial is considered a match if an inclusion criteria leaf nodes on one matched paths are true and none of an exclusion criteria leaf nodes on the path are true.

18. The computer program product of claim 15, wherein searching the one or more proposed clinical trials is based on standardized disease codes and relationship among the standardized disease codes.

19. The computer program product of claim 15, wherein determining further comprises:
program instructions to retrieve clinical trials from a clinical trial database that matches a patient clinical information without finding an exact match, using a standardized disease condition codes and relationships to provide a of matching clinical trials.

20. The computer program product of claim 15, wherein determining further comprises using natural language processing to extract data from both the one or more proposed clinical trials and the patient clinical information to match the patient with the clinical trial traits that fit the patient clinical information needs, wherein patient clinical information comprise: type of illness, allergies, biomarker data, type of disease, length of illness, length of disease, cause of illness, cause of disease, medical history, family medical history, gender, physical fitness, age, socioeconomic status, nationality, genetic make-up, genetic response to medication, and genetic predispositions.
